(19) United States
(12) Patent Application Publication
(10) Pub. No.: US 2011/0076316 A1
(43) Pub. Date: Mar. 31, 2011

(54) SCALABLE MATRIX FOR THE IN VIVO CULTIVATION OF BONE AND CARTILAGE

(76) Inventors: Sureshan Sivananthan, London (GB); Patrick Hans Warnke, Robina (AU); Eugene Sherry, New South Wales (AU); Krish Gopalakrishnan, Singapore (SG)

(21) Appl. No.: 12/682,218

(22) PCT Filed: Oct. 8, 2007
(86) PCT No.: PCT/MY2007/000066

§ 371 Date: Dec. 13, 2010

(51) Int. Cl. A61K 35/12; A61F 2/02; A61F 2/28; A61K 9/00; C12N 5/071; A61K 38/02; A61K 38/18; A61K 38/48; A61P 19/02; A61P 19/08; A61P 43/00; B29C 35/08

(52) U.S. Cl. 424/423; 623/14.12; 623/16.11; 424/93.7; 435/395; 514/2.3; 514/8.9; 424/94.63; 264/400

(57) ABSTRACT

The present invention provides implantable receptacle devices (and methods) for use in bone and tissue regeneration which provide immediate structural stability and strength to a zone where tissue regeneration is required. By virtue of their size, shape and construction, the devices are scalable, modular, structurally stable, self-stacking in three dimensions, can be aggregated to an anatomically accurate shape, and hold various materials delivered into the implant area so as to create a highly regenerative micro-environment. They can be implanted via less invasive surgical procedures, and because they act as external scaffolding as well as being imbedded as an integral part of a matrix for the effective and rapid regeneration of bone and cartilage in vivo, they may provide significant advantages to patients or subjects in terms of reduced pain, faster healing and fewer complications.

### FIELD OF INVENTION

The present invention relates to an implant system for the in vivo regeneration of stable bone and cartilage, and in particular to devices specifically shaped as receptacles for scaffold constructs which together form a stable matrix for the regeneration of bone and cartilage in vivo.

### BACKGROUND OF THE INVENTION

Bone loss is a major problem in trauma and orthopaedic surgery. Everyday, surgeons have to deal with the challenge of patients with major bone loss, either due to trauma, cancer, congenital defects, previous surgery or failed joint replacements.

Bone tissue is composed of a matrix that primarily consists of collagen protein, but is strengthened by deposits of calcium, hydroxyl and phosphate salts, referred to as hydroxyapatite. Inside and surrounding this matrix lie the cells of bone tissue, which include osteoblasts, osteocytes, osteoclasts and bone-lining cells. All four of these cell types are required for building and maintaining a healthy bone matrix, as well as remodelling of the bone under certain conditions.

Most importantly, bone is an extremely dynamic and well organised tissue, from the modulation of the hydroxyapatite crystal arrangement at the molecular level to the strain pattern of the trabecular network at the organ level. The synergy of the molecular, cellular and tissue arrangement provides a tensile strength comparable to that of cast iron, with such an efficient use of material that the skeleton is of surprisingly low weight for such a strong supporting structure.

At the microscopic level bone consists of 2 forms: woven and lamellar. Woven bone is considered immature bone and is usually found in the new-born or in fracture callus (healing bone). Lamellar bone is more organised and begins to form 1 month after birth. Thus, lamellar bone is a more mature type of bone that results from the remodelling of immature woven bone. The highly organised, stress oriented collagen fibres of lamellar bone give it anisotropic properties—that is, the mechanical behaviour of lamellar bone differs depending on the orientation of the applied force, with its greatest strength parallel to the longitudinal axis of the collagen fibres.

Injury, disease and developmental defects can all result in bone defects that require bone grafting procedures, where new bone or a replacement material is placed in apertures around a fractured bone, or in bone defects. Bone grafting allows bone healing by filling the gap, or merely provides mechanical structure to the defective bone, through the provision of artificial material that is not incorporated into a patient's own bone.

Autograft may be used where it is appropriate to take the patient's own bone tissue from another site in the body, usually the iliac crest, although bone from the distal femur, proximal tibia or fibula may also be used. Autograft has advantages: it provides osteoconductivity (i.e., the graft supports the attachment of new osteoblasts and osteoprogenitor cells). Furthermore, it provides osteoinductivity, or the ability to induce non-differentiated cells into osteoblasts.

In the context of autograft for injuries such as bone fractures, the grafting procedure can be quite complex, and may fail to heal properly. Grafting for bone fractures is generally only considered when a reasonable sized portion of bone has been lost via fracture. In this context, bone grafting may be performed using the patient's own bone, usually taken from the iliac crest, or using bone from a donor (allograft). The replacement bone is usually held in place by physical means (e.g., screws and pins), while the healing process occurs.

The drawbacks for autograft procedures include surgical complications (e.g., acute and chronic pain, inflammation, infection), and limitations in relation to the amount of bone that can be harvested for grafting. Furthermore, complications occurring after bone grafting include fracture at the donor site after cortical graft removal, intra-operative bleeding and postoperative pain after iliac crest biopsy and stress fractures, hernias through an iliac donor site and gait problems.

The alternative procedure, allograft, where bone graft material is taken from a donor or cadaver, offers some advantages over autograft in terms of the lack of surgical complications in obtaining the bone graft material. However, there is a risk of disease transmission from the donor to the recipient of the bone graft material, which is not overcome by pre-implantation treatment of the tissue with techniques such as gamma irradiation. Furthermore, the allograft may not knit well with the patient's own bone, leading to weakness at the point of union of the graft. Also, where bone is harvested from a donor, there exist the same risks as harvesting replacement bone from the patient, as discussed above.

A variety of alternative graft materials exist, including ceramic materials, polymeric materials and chemically inert substances. Many of these are commercially available. These bone substitutes are often inoculated with bone marrow and/or growth factors to provide the osteoconductivity and osteoinductivity that is seen when autograft bone is used.

In the case of certain bone substitute materials, there is the disadvantage that they do not become permanently incorporated into a patient's own bone and are thus subject to breakage, loosening and erosion.

While bone grafting using a polymeric matrix or bone graft has been found to have the capacity for bone regeneration (Borden et al., J Bone Joint Surg Br. 2004 November; 86(8):1200-8; Mankani et al., Biotechnol Bioeng. 2001 Jan. 5; 72(1):96-107), the site of regeneration will naturally be in a weakened state until full bone mineralisation and osteoblast replacement is attained.

Extracellular matrices for example hydroxyapatite, various metals like magnesium, tantalum or titanium, calcium sulphate, tricalcium phosphate and various polymers have been used for a long time to act as scaffolds, alone or in various combinations and sub-combinations, to facilitate tissue engineering of bone and improve the success of bone grafting procedures. One recent example of prior art in regard to extracellular matrices is U.S. Pat. No. 7,201,917, which also contains numerous references to prior art in the field. The most common disadvantage of these scaffolds, as well as methods of bone grafting, is that the process of healing (repair) or incorporation of the new bone takes weeks or sometimes months; and in that interim period the newly formed bone is subject to breakage, erosion or damage.

As is well known to those experienced and practised in orthopaedic surgery, an additional drawback common to all these grafting procedures is that bone graft or bone graft substitute, when used to fill a defect or gap or space is not as strong as normal bone and therefore needs to be supported by or augmented with an internal or external fixation until healing and remodelling occurs.

Methods for the manipulation of scaffold pore size, porosity, and interconnectivity are considered extremely important to the science and art of bone and tissue regeneration (Ma and Zhang, 2001, J Biomed Mater Res, 56(4):469 477; Ma and Choi, 2001 Tissue Eng, 7(1):23 33). An extensive review of the state of the art in orthopaedic implants and commercially available products reveals that pore size, material, preparation methods and chemical treatment are extensively manipulated in an effort to increase the chances of rapid growth, healing and/or regeneration. However, whether for bone grafts/substitutes or for other scaffolding, the possibility of providing “imbedded” structural stability and strength to scaffold materials, or interior reinforcements analogous to reinforced concrete, at an intermediate scale within an implant zone has not been considered adequately.

Numerous other publications and patents have been filed with respect to implants, implant materials and implant design, all addressing the issues within bone and tissue engineering as they are currently understood. In particular, two items of prior art which address unusually shaped “plugs” for orthopaedic use are U.S. Pat. No. 5,861,043 and WO 01/91672 A1. The devices described in these disclosures are principally void-filling plugs which may have different possible shapes. While they provide some structural stability, they are in the main soft and not as hard as cancellous or cortical bone. In fact they should have the same Young's modulus as hyaline cartilage and/or subchondral bone or they would not work for the purpose they are designed for. In addition, methods of keeping these conjoined or aggregated may be complex or unreliable in surgical practice. Moreover, they are not designed to allow vascularisation or angiogenesis to occur with ease in spaces filled by these plugs. Consequently there still remains a need for improved devices and methods for the genuine regeneration of bone and cartilage, in a manner that is better customised and optimised for the individual patient, and preferably in vivo.

In a departure from the above approaches, the devices of our present invention are designed as complex receptacles which self-stack when juxtaposed or pressed together, and reinforce smaller-scale scaffolding in an effective manner while tissue regeneration and healing take place.

Recently there have been reports (Brown RA et al., Advanced Functional Materials. 2005;15:1762-1770) of ways in which to speed up controlled engineering of biomimetic scaffolds by rapid removal of fluid from hyperhydrated collagen (or other) gel constructs using plastic compression technology. The huge scale shrinkage in the process allows the introduction of controllable mechanical properties to the construct. Critically, this process takes minutes rather than the conventional days (or weeks) normally necessary to engineer collagen tissue. However, this technology at present can only be used in vitro to produce native collagen structures with controllable nano- and micro-scale biomimetic structures. There is a need for creating a reliably osteoinductive and osteoconductive biomimetic construct fabricated by plastic compression which may be implanted. The product of this plastic compression technology still needs to be delivered viably into living systems, and a device or method is required for expanding or scaling it up to dimensions much larger than currently achieved, stabilising it in three dimensions and creating the appropriate biomechanical and cellular environment to enable remodelling and healing. In general, a need also exists for delivering other matrices or scaffolding constructs viably into living systems and expanding or scaling these up to dimensions much larger than currently achieved, stabilising them in three dimensions and creating the appropriate biomechanical and cellular environment to enable remodelling and healing.

### OBJECTS OF THE INVENTION

It is an object of the present invention to provide devices (and methods) for use, in bone and tissue regeneration which provide immediate structural stability and strength, create a highly osteoinductive and osteoconductive micro-environment, can be quickly and rapidly scaled up by those practised in the art to a desired shape and dimension, can be implanted via less invasive surgical procedures, and possibly provide significant advantages to patients or subjects in terms of reduced pain, faster healing and fewer complications.

It is another object of the invention to improve postoperative results following reduction and treatment of spinal fractures using minimally invasive techniques.

Further objects hereof are extant although not described.

### SUMMARY OF THE INVENTION

The present invention provides a bone and tissue regeneration system, which combines:
A. an implantable device at meso-scale, specifically shaped and designed as a receptacle for
B. biomimetic constructs at nano- and micro-scale, and
C. if necessary an exterior hull or wrapper or mesh at macro-scale that may carry, contain or encapsulate the above mentioned devices and constructs

The basic concept is that there are at least 2 orders of scaffolds:
(A) a strong, structurally stable, specifically shaped scaffold device to take weight/load and provide compression resistance, and
(B) a highly osteoinductive and softer scaffold which makes rapid bone or cartilage ingrowth possible.

The implantable meso-scale devices of (A) provide a meso-scale scaffold, and we define “meso-scale” herein as being in the dimension range of one or more micrometres up to tens of millimetres. These implantable devices have been shaped and designed with two purposes:
1. providing the necessary immediate structural strength and stability to the implant zone within the mammalian body where bone or tissue regeneration is required.
2. providing the nano- and micro-scale biomimetic scaffolds or constructs of (B) with a complex interlinked receptacle within which these biomimetic constructs can be juxtaposed or connected together, and grow so that in their final form and position the meso-scale device(s) could be seen as imbedded within, and reinforcing the biomimetic scaffold.

Thus the primary but non-limiting purpose of this invention is to form a scalable matrix for the regeneration of bone or cartilage within a mammalian subject. We recognise, however, that the principles described herein may be used for the regeneration of other tissue as well. This meso-scale matrix is intended for intra-osseous space or intra-cartilage space, but may be used elsewhere and in other applications in a mammalian body. The devices providing meso-scale scaffolding possess specific shape(s) and are designed to aggregate or stack into stable interconnected meso-scale receptacles for biomimetic constructs, thus providing scalable osteoconduction within the implant zone.

Further, when a meso-scale scaffold device and biomimetic constructs are combined together and seeded or infused with various cells and growth factors, they form a highly effective, scalable, customised in vivo regeneration matrix.

In its most basic embodiment, therefore, the meso-scale scaffold protects the inner softer scaffold until bone or tissue growth is strong enough. The meso-scale scaffold may or may not be removed at a later date. Moreover, inner biomimetic scaffolds may be loaded into meso-scale scaffolds before, during or after surgery.

We also provide here some non-limiting methods for combining and/or aggregating such scaffolds and constructs as well as an exterior hull at several levels of structure and dimension to create a complete bone or cartilage regeneration matrix which is customised and optimised for the individual patient.

In one aspect, this invention consists of a meso-scale device which when combined with biomimetic constructs by one skilled in the art, is capable of causing clinically significant levels of bone or cartilage regeneration within a patient. In another aspect, this invention comprises the method for combining and deploying the above-mentioned devices and constructs either in preparation for, and/or during surgery so as to cause clinically significant levels of bone or cartilage regeneration within a patient.

The implantable receptacle devices, by virtue of their size, shape and construction, have the following properties: they are scalable, modular, structurally stable, self-stacking in three dimensions, can be aggregated prior to or during surgical procedures to an anatomically accurate shape, provide structural integrity to a zone where tissue regeneration is required, are capable of holding and interconnecting various constructs, materials, and biomolecules delivered into the implant area, and act as external scaffolding as well as being imbedded as an integral part of a matrix for the effective and rapid regeneration of bone and cartilage in vivo.

It is the shape(s) of the scaffold device/components which fulfils many of the various functions which are described herein, and makes possible the various properties of the bone and tissue regeneration system. For example, in one non-limiting approach, the biomimetic constructs made by plastic compression may be micro-manipulated into a stable position and conformation/orientation within the single compartment of the meso-scale scaffold device. When these meso-scale scaffold devices then stack together in 3-dimensional space, their internal compartments are all interconnected in a stable structure which resists deformation, and create a complex interconnected receptacle extending the biomimetic constructs within the intra-osseous or intra-cartilage space, thus allowing cell-mediated remodelling of bone or cartilage tissue to take place throughout the implant area.

### DETAILED DESCRIPTION OF THE INVENTION

The present invention arose from the development of devices or a system for treating fractures of the spine or other bone that provides a bone conserving or bone preserving approach and can be done using minimal invasive instruments. It can however be extended as a solution for other tissue as well.

The term “patient” refers to patients of human or other mammal origin and includes any individual it is desired to examine or treat using the device(s) of the present invention. However, it is understood that “patient” does not imply that symptoms are present. Suitable mammals that may benefit from use of the device include but are not restricted to, humans, primates, livestock animals, laboratory test animals, companion animals (eg. cats and dogs) and captive wild animals.

Description of Meso-Scale Scaffolding

The inventors have noted from the literature and personal experience that most bone substitute materials available today, although good at providing osteoconduction (and osteoinduction in some cases), lack the necessary strength to withstand compressive and other forces. Once used, most of these materials do not have the anisotropic properties of bone until healing occurs—a process which takes 6 to 8 weeks.

Certain specific shapes, when applied to bone substitute material, metal or plastic (particularly those made using existing SLM or SLS technology) gain compressive strength through stacking. These shapes may be broadly described as polyhedral. They also self-stack, which we herein define as the tendency to form a stable conjoined structure when aggregated together in close proximity in 3-dimensional space. This property is also present in nature and allows seemingly small discrete structures to build into larger robust structures. However, most polyhedral shapes have been described in Euclidean and other geometry but are seldom found in nature. Moreover, it has hitherto been neither obvious nor simple to fabricate such shapes from available materials. The present invention demonstrates the actual fabrication by SLM of polyhedral shapes that are small, stable, can be easily stacked together and possess several other properties more fully described below.

The primary device, which is a unit of the final meso-scale scaffolding system is a polyhedral receptacle. Those well-versed in this area of mathematics and geometry will know that the term polyhedron may be defined as a three-dimensional object composed of a number of polygonal surfaces, which includes but is not limited to all polyhedra described as Platonic, Archimedean, Kepler-Poinsot, having Tetrahedral/Octahedral/Icosahedral symmetry, Non-Convex Snubs, Prisms/Antiprisms, Johnson Solids, Near Misses, Stewart Toroids, Pyramids and Cupolae, and Degenerates as well as the compound and/or stellated versions of all the aforementioned, including also geodesic spheres, geodesic domes or sections of geodesic spheres and domes.

In particular, of all the known polyhedra, some highly preferred shapes are the dodecahedron, the hexagonal prism, the hexagonal antiprism, the pentagonal dipyramid and the tetrahedron (See FIGS. 1-5).

In a highly preferred embodiment of these and other shapes, the polyhedra are “wireframe”; we define “wireframe” hereafter for the purpose of this invention as follows: an accurate description of a “wireframe” meso-scale polyhedral scaffolding device is that the substance/material of construction of the polyhedron resides only along the edges encompassing each polygonal face of the polyhedral shape; the rest of the polyhedral shape is empty or hollow and can be filled with other substances. In other words, they are polyhedral receptacles. Expressed another way, the ratio of space to substance in these “wireframe” polyhedra is in excess of 80:20 (FIGS. 1-6). However, as described below, other embodiments may not have the same space: substance ratio.

In another preferred embodiment, the polyhedron may be partially “filled” in any manner by its material of construction, rather than be completely “wireframe”; for example, a dodecahedral shape may appear to be part “filled” with its own material of construction in any manner desired; or it may have some of its faces removed to create a “basket” (see FIG. 7). In an obvious variation of these embodiments, the polyhedron may be completely “filled” by its material of construction but be porous in nature, and/or adsorptive or absorbent in function.

In another highly preferred embodiment, by virtue of their material of construction, the polyhedra may be either “wireframe” or full-face, and may be first unfolded to a flattened, planar, polygonal shape; and/or they may be folded from this flattened planar polygonal shape to any other complex topology or shape by several random or directed folds, all with the purpose of minimally invasive surgical implantation. In one non-limiting example of this embodiment, the polyhedra are constructed from nitinol, which confers many of the above properties on the device. Thereafter, the polyhedra thus treated may be left in any shape or topology, or re-folded to their original shape, and this process may be carried out before, during or after the surgical implantation, so that the meso-scale scaffold(s) perform their required function in the regeneration matrix (as described in the section, “Construction of a Scalable Tissue Regeneration Matrix”, below). One non-limiting version of the step-wise unfolding and refolding is depicted in FIG. 9.

In a variation of the preceding embodiment, by virtue of their material of construction, the polyhedra may be significantly and reversibly compressed to a much smaller volume with the purpose of minimally invasive surgical implantation, and thereafter may be caused to regain their original dimensions and shape during or after the implantation to perform their required function in the regeneration matrix (as described in the section, “Construction of a Scalable Tissue Regeneration Matrix”, below).

In one non-limiting example of a typical method of use of all the above embodiments, a composite or aggregate formed of a multitude of discrete polyhedra, which can each be any size upwards of 1 micrometre in any one dimension, and stacked together in 3-dimensional space, forms the required interior scaffolding or reinforcement within a given bone or cartilage undergoing repair or regeneration; in other words, this composite of discrete stacked polyhedra fills out the intra-osseous or intra-cartilage space where repair or regeneration is needed (See FIG. 6).

Obviously, any assortment of polyhedra in a single or multiple shapes, whether solid, partially filled or hollow, using any appropriate metal, plastic, polymer, or other material capable of retaining 3-dimensional shape, and in any assortment of sizes ranging upwards from 1 mm in any one dimension, may be packaged together into a kit which allows a surgeon skilled in the art to select the exact size, dimensions, shape and scale of meso-scale scaffolding required by a patient for surgical implantation.

In the most preferred process, the polyhedra of the aforementioned embodiments are fabricated by selective laser melting (SLM). See FIGS. 8a-8d, which show photographs of an SLM plate prior to excision of very small polyhedra (1.5 mm-2.1 mm). However, the polyhedra may also be formed by other methods and processes of solid fabrication, rapid prototyping (particularly selective laser sintering or SLS), or extrusion, or nano-assembly, or nano-construction, or gel formation and hardening etc.

Construction of a Scalable Tissue Regeneration Matrix

In a preferred embodiment and related method of constructing such an embodiment, the (single) compartment(s) found within each of the “wireframe” polyhedra or partially “filled” polyhedra are loaded with collagen sheets assembled into spirals, formed by the process of plastic compression. These collagen spirals themselves are known to contain biomimetic structures at nanometric and micrometric scale (Brown RA et al., Advanced Functional Materials. 2005;15:1762-1770).

In a preferred variation of the above embodiment, the collagen sheets or micro-spirals manufactured by plastic compression are first seeded with any combination of biologically functional cells, such as but not limited to stem cells, fibroblasts, osteoblasts, osteocytes, chondrocytes etc., and/or other materials such as fibronectin or hydroxyapatite or other polymers; and then in one preferred process, this composite collagen construct is loaded within the polyhedral compartments and then cell-cultured in vitro; and in another preferred process, this composite collagen construct is loaded within the polyhedral compartments and implanted surgically to allow remodelling and healing entirely in vivo.

In yet another preferred process, any of the above embodiments or constructs may be perfused, injected, seeded, or washed or filled with biologically functional cells such as but not limited to fibroblasts, osteoblasts, osteocytes, chondrocytes, soft tissue cells, endothelial cells, blood cells, immune cells or stem cells, (whether autologous or exogenous), and/or preparations of biomolecules such as growth factors (e.g., TGF-β superfamily, BMP-1, etc.).

In one preferred variation, any of the above embodiments of the tissue regeneration matrix may be coated with antimicrobial peptides or other drugs and medications. In another preferred variation, any of the above embodiments of the tissue regeneration matrix may be used as a delivery system or vehicle for the emplacement of slow-release drugs or other bioactive molecules.

In another preferred embodiment, any aggregation of polyhedra at any level and in any shape, and in any of the embodiments described above, may be wrapped in a polymer, preferably biodegradable, so as to enable the entire construct to be delivered into an intra-osseous, subperiosteal or bone surface zone or cartilaginous zone to promote bone or cartilage regeneration.

In all these embodiments, their variations and through the accompanying methods and processes, the polyhedra provide structure and stability at meso-scale, from ten(s) of micrometres to several tens of millimetres. Thus the collagen-loaded polyhedra become a significant enabler of tissue regeneration at multiple scales: nano-, micro- and milli-. Since the polyhedra can themselves be manufactured in various sizes, and also stacked, the entire tissue regeneration system of the present invention is highly and precisely scalable in the hands of a surgeon skilled in the art.

The inventors view this special combination of scalable and stackable polyhedral receptacle devices, biomimetic collagen constructs and cells/growth factors as a true tissue regeneration “matrix”, as distinct from an inert or biologically inactive scaffold. Since the nano- and micro-scale structures of the plastic-compressed collagen spirals are held in extensively interconnected compartments in 3-dimensional space by wireframe polyhedra, they can be scaled outwards or expanded in three dimensions and stacked stably within the intra-osseous or intra-cartilage space in a manner which allows perfusion with fluids, media, gels, blood and filling with any other materials of choice. Thus this invention is designed to maximise osteoinduction, osteoconduction, osteogenesis and the chances of angiogenesis/vascularisation, extensive cellular remodelling and the ultimate healing of the bone or cartilage in vivo.

Materials and Nature of Construction of the Scalable Tissue Regeneration Matrix

This scalable matrix, particularly the exterior hull and meso-scale scaffold devices, may be fabricated from a wide range of clinically approved or accepted biocompatible materials, such as metals and their alloys (titanium, cobalt chrome, stainless steel, nitinol, etc.), ceramics (hydroxyapatite or tricalcium phosphate) or polymers (polylactide, polyglycolide, polyetheretherketone, etc.), or bioactive glasses (Bioglass, Biogran etc.), or any combination of these or other materials which may be approved for such uses. The materials may be combined so as to allow the polyhedral receptacles to either remain implanted and inert, or degraded by natural processes, or allow them to be completely or partially resorbed into the mammalian body.

In one preferred embodiment, the meso-scale receptacle devices may be aggregated into a kit comprising an assortment of polyhedra fabricated from a single material. In another embodiment, the meso-scale devices may be aggregated into a kit comprising an assortment of polyhedra fabricated from different materials. In yet another embodiment, the polyhedra may be fabricated from one material but loaded, embedded, packed, coated, lined or infused with one or more other materials to confer upon the stacking structure a plurality of osteoinductive and osteoconductive properties. All these embodiments may be presented variously alone or in combination in a multitude of commercially available kits.

In yet another preferred form, some components of the matrix may be inserted into the polyhedra in gel or semi-fluid form, which can then harden when they are activated by a UV light or other similar light source.

In yet another preferred form, any or all of the above embodiments of the scalable tissue regeneration matrix may be constructed of or include porous materials, or deliver such materials into the zone where bone and cartilage regeneration is required.

In still another preferred form, any or all of the above embodiments of the scalable tissue regeneration matrix may be nano-assembled, or nano-textured or nano-surfaced by methods known to those skilled in the art so as to further enhance the osteoinductive and osteoconductive properties of the scalable tissue regeneration matrix.

Properties, Features and Benefits of the Scalable Tissue Regeneration Matrix

Preliminary and simple studies and fabrications by the inventors have shown that the polyhedral shape of the meso-scale scaffold, particularly when made at millimetric scales by SLM, has several properties and features:
1. The polyhedra ‘flow’ as a series of discrete particles when pushed through MIS channels into a surgical (fracture or bone defect) site, or through any of the mammalian body's own channels, spaces or vessels.
2. They self-stack in three dimensional space to fill out or form any shape which is robust, i.e., resists deformation, provides immediate structural integrity and helps load-bearing.
3. By aggregating/stacking within larger polyhedra, they can be scaled upwards either continuously or step-wise into dimensions of a few cubic centimetres. In one non-limiting example, there can thus be multiple sizes, and multiple types of polyhedrons within the same construct. In one non-limiting example, a large icosahedron at 8 mm could contain or be packed with several dodecahedrons at 2 mm.
4. They can be stacked easily during surgery within any existing or created void, aperture or gap in bone or tissue structure by the surgeon using visualisation aids (for example image intensifiers, endoscopy and fluoroscopy).
5. They can also be aggregated and stacked prior to insertion or implantation into a fracture or defect site.

The meso-scale scaffolds have several benefits:
they decrease or eliminate the need for artificial void creation within a fracture zone, as the scaffolds act as an imbedded, internal sub-structure stacking around and holding the bone fragments together, and/or translating and elevating compression fracture zones and encourage bone healing.
they increase the stability and structural integrity within fracture zones as well bone graft sites due to their ability to interlock at various levels.
enable high, precise control of fracture reduction particularly in small bones and intra-osseous damage zones.
no increase in internal tissue pressure or aggravation of the molecular/immunological stress which accompanies cell damage.
low chance of diffusion, migration, dislodgement or deformation after surgery
increased chances of angiogenesis due to the higher proportion of available or “empty” space in the construct.
increased chances of cell-mediated remodelling.

Description of Exterior Hull

The exterior hull may be either a mesh-like or lattice-like reticulated single construction, made by any method of fabricating solids, and may encapsulate, surround, circumscribe, be adjacent to, or contiguous with the fracture zone, bone defect or bone loss area where structural integrity is needed and bone repair or regeneration are to be carried out.

In a most preferred embodiment, the exterior hull is built to the anatomically accurate shape of the bone or cartilage which is to be repaired or regenerated, in the precise dimensions and orientation required by the patient requiring such repair or regeneration. In a preferred method for making the above embodiment, the exact shape and dimensions of the required bone or cartilage are obtained from X-rays or 3D CT scans of the patient, or other similar imaging technology such as MRI or PET scans etc., which may be readily available, and the exterior hull is customised to the exact shape required using computer design or CAD software.

One major design variation of the above embodiment is that on its inner surfaces, the exterior hull may be inlaid with polyhedral recesses or “niches” capable of receiving and holding aggregations or stacks of meso-scale scaffolding devices in a stable position.

The exterior hull is made as a single free-form entity without the need for joining or articulating separate pieces. In a particularly preferred process, the external scaffolding is made by selective laser melting (SLM). It may also be formed by other methods and processes of solid fabrication, rapid prototyping, or extrusion, or gel formation and hardening etc.

In another preferred embodiment the exterior hull may be soft and pliable and be made of a sheet of polyglycolic acid or polycaprolactone or collagen or any combination or sub-combination of these and other biomimetic substances.

### BRIEF DESCRIPTION OF THE DRAWINGS

FIG. 1a: Schematic drawing of a dodecahedron as wireframe

FIG. 1b: Unfolded net of dodecahedron

FIG. 2a: Schematic drawing of a hexagonal prism as wireframe

FIG. 2b: Unfolded net of hexagonal prism

FIG. 3a: Schematic drawing of a hexagonal antiprism

FIG. 3b: Unfolded net of hexagonal antiprism

FIG. 4a: Schematic drawing of a pentagonal dipyramid

FIG. 4b: Unfolded net of hexagonal antiprism

FIG. 5a: Schematic drawing of a tetrahedron

FIG. 5b: Unfolded net of hexagonal antiprism

FIG. 6a: Several “filled” dodecahedra stacked together in 3 dimensions

FIG. 6b: Wireframe view of stacked dodecahedra

FIG. 7: A partially “filled” dodecahedron with interior compartment

FIG. 8a -8d: Photographs of an SLM plate showing rows of built polyhedra prior to excision or harvest

FIG. 9: Unfolding of a dodecahedron into a flat polygonal planar shape and step-wise re-folding into a dodecahedron

FIG. 10: View of the Ilium and its structure

FIGS. 11a and 11b: Bone harvest zone on the ilium, and area to avoid

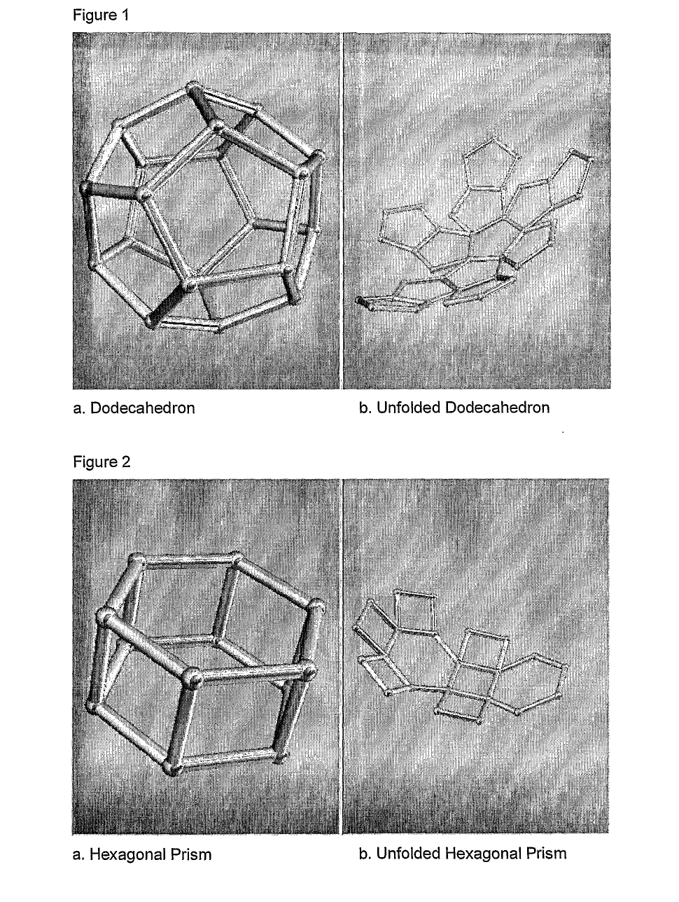

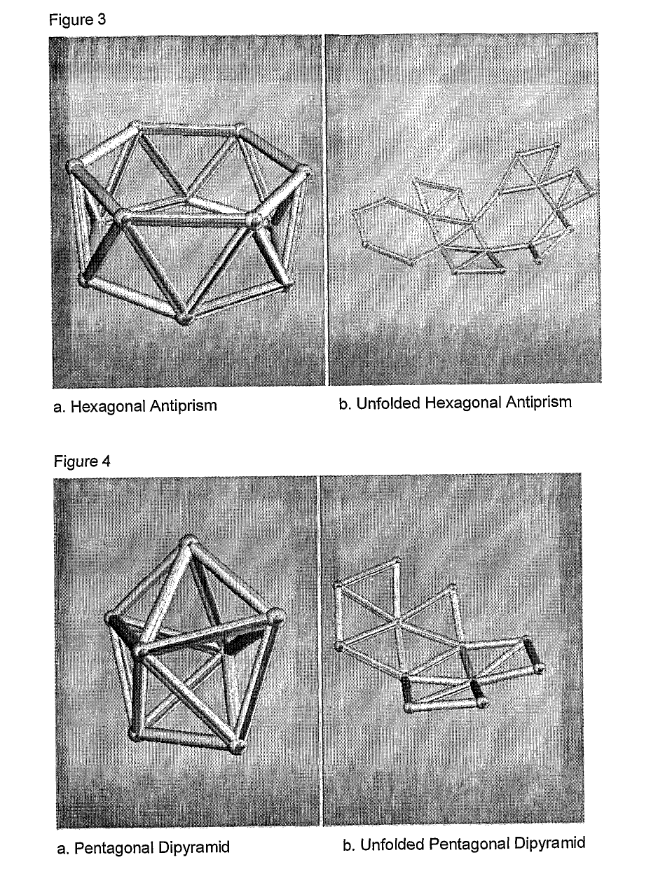

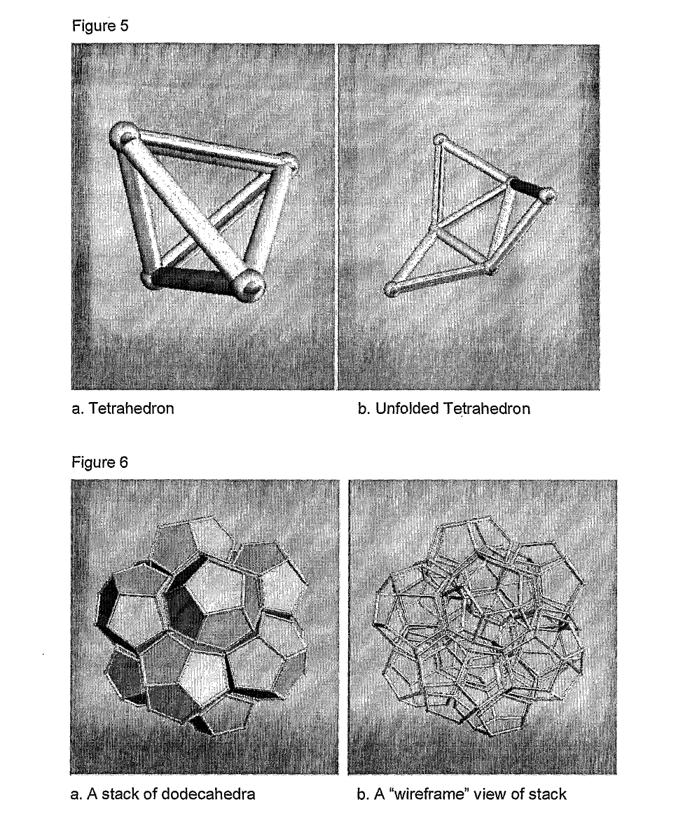

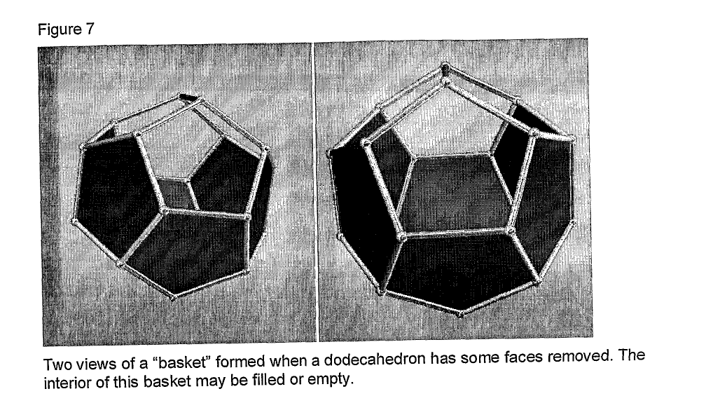

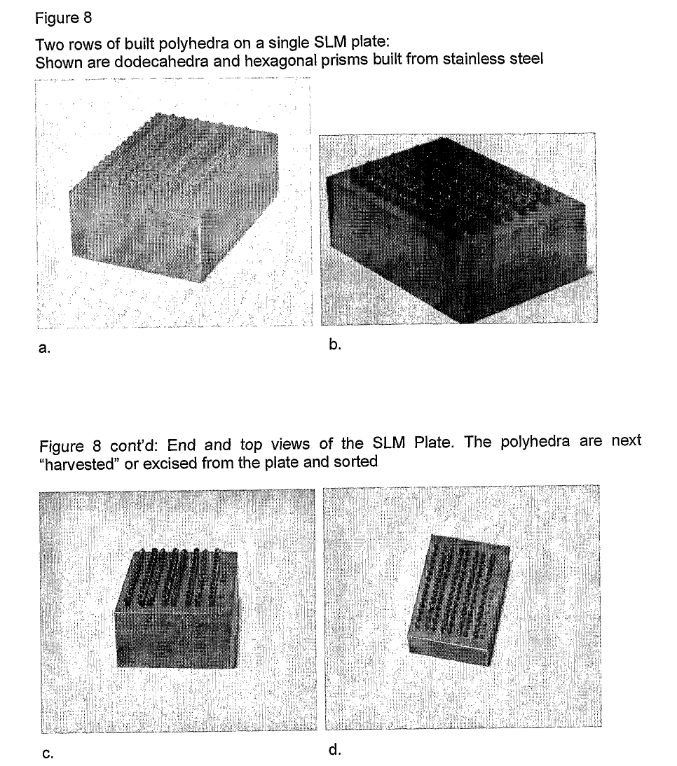

### METHODS AND EXAMPLES OF USE

The use, application and methods pertaining to the scalable matrix will be further understood by reference to the following non-limiting examples:

### Example 1

### Treating Lumbar Compression or Burst Fractures

The traditional way of treating these fractures is to perform a Vertebroplasty or Kyphoplasty in the case of compression fractures and in the case of burst fractures of the spine requiring surgical intervention to achieve biomechanical stability, to perform a combined anterior instrumentation and short segment posterior instrumentation (SSPI). In low grade burst fractures, Vertebroplasty plus SSPI may provide a less invasive method of stabilising the burst fracture but there have been no conclusive tests or patient trials showing that this method is stable. Moreover there is a risk of cement or existing bone substitute materials leaking out and injuring the spinal cord, nerves or blood vessels.

It is important to note that vertebral burst fractures are typically associated with high impact axial loading resulting from trauma.

Surgical Instructions

Step One

Place the suitably consented and anaesthetised patient prone on a montreal mattress.

Step Two

Reduce the fracture and stabilise using Short Segment Posterior Instrumentation of your choice. The rods will bridge the fractured vertebra.

Step Three

Make sure the spinal canal is adequately decompressed and remove any loose bone fragments.

Step Four

Option 1

Stack or pack spaces in the fractured vertebra with the scalable matrix, inserted through the pedicle allowing the matrix to do its job and create a stable interlock. This is done under fluoroscopic control.

Option 2

Stack or pack spaces in the fractured vertebra with the scalable matrix, inserted through the extra-pedicular approach allowing the matrix to do its job and create a stable interlock. This is done under fluoroscopic control.

Step Five

Once a stable construct is obtained, wash, obtain haemostasis and close in layers. Use a redivac drain for 24 hours.

### Example 2

### Correction of Various Structural Defects

a. Fill the defects in the talar dome of the ankle following post traumatic osteochondral fractures where there is a large hole. Scalable matrix is filled into the curetted holes.

b. Fill the defects in surface of the knee where there are defects/holes following osteochondritis dissecans. Place the matrix into the curetted holes.

c. Fill the defects in the mid portion of the scaphoid bone where there is an established non-union with a large defect which needs filling before a screw is placed.

d. Following avascular necrosis of the femoral head there is a large cavity which could be filled with the matrix prior to placing a re-surfacing metallic femoral head.

### Example 3

### Maxillofacial Surgery

In maxillofacial surgery augmentation procedures, the scalable matrix could be used. One particular example is sinus floor augmentation; however all bone cavities such as those from tooth extractions, cysts, fractures or defects after tumour removal can be filled using the scalable matrix.

### Example 4

### Bone graft harvesting

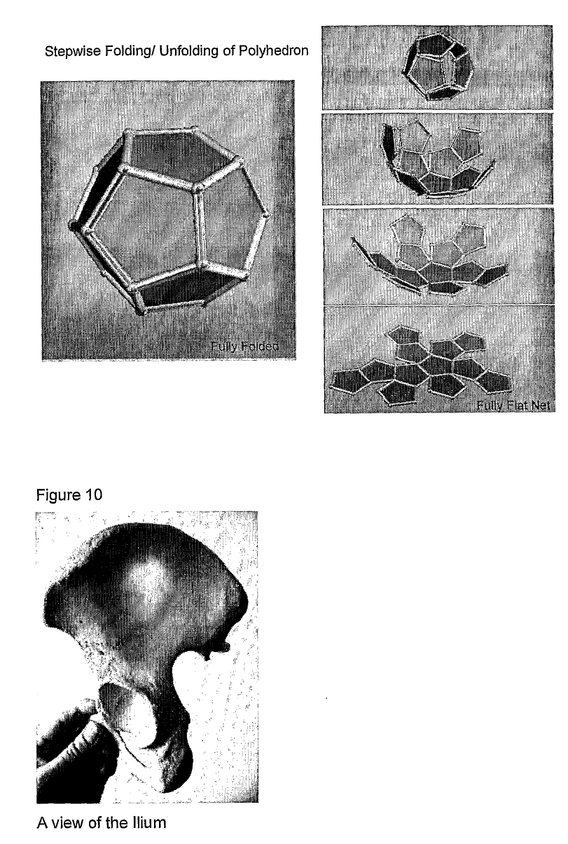

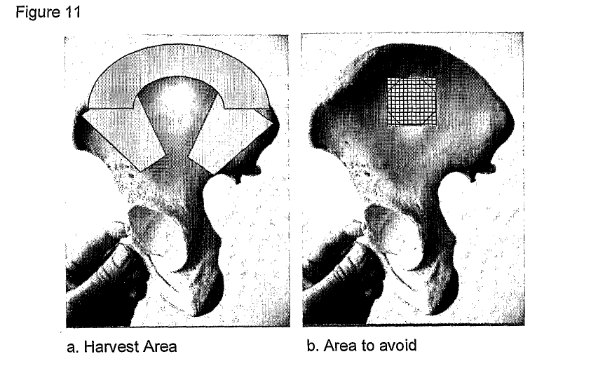

The traditional way of harvesting bone graft from the pelvis is associated with a high complication rate. The reason is that the graft is taken by an incision over the iliac crest with a vertical segment of iliac bone removed. But apart from the region of the ASIS and PSIS the ilium is tissue-thin here (FIG. 10), and post-harvest, it bleeds and collapses. The bulk of iliac bone is found just below (2 cm) and parallel to the iliac crest; by the ASIS and the PSIS. This is where the bone graft should be harvested; the hatched area should be avoided (FIGS. 11a and 11b).

Surgical Instructions

Step One

Place the patient prone/supine (face down or face up) or lateral (on their side)—surgeon's preference. Place a 1 cm incision below the outer prominence of the PSIS or ASIS. Place the guide though small bony entrance, parallel to crest and radiate downwards from ASIS or PSIS.

Step Two

Pass bone harvester subperiosteally over guide a distance of up to 5 cm.

Remove the cuttings. (OR pass the dowel cutter over the guide wire.

Cut dowels in multiple directions.

Remove the dowels).

Step Three

Place a suction catheter down the channel/dowel the holes and suck out the bone marrow including stem cells.

Fill gap with bioabsorbable space material.

Step Four

Option 1

Pass a bougie down the dowel holes and expand the periosteal sleeve (there is the option to pre-contour the matrix). Then pack the dowel holes with Scalable Matrix.

Option 2

Pass the bougie shaped as tibial shaft bone, femoral head, lower femur, upper tibia, proximal humerus, then expand, then pack with Scalable Matrix.

Harvest when mature bone formed (assess either by X-ray, bone scan or biopsy).

While all the disclosures herein are susceptible to various modifications and alternative forms, specific exemplary embodiments of the invention have been shown by way of example in the drawings and have herein been described in detail. It should be understood, however, that there is no intent to limit the disclosure to the particular forms disclosed, but on the contrary, the intention is to cover all modifications, equivalents, and alternatives falling within the spirit and scope of the disclosures. Various combinations and subcombinations and features may be practiced with or without reference to other combinations, subcombinations and/or features, alone or in combination, in the practice of the invention, and, moreover, numerous further adaptations and modifications can be effected within its spirit, the literal claim scope of which is particularly pointed out as follows.

There are a plurality of advantages of the present disclosure arising from the various features of the devices, kits and methods described herein. It will be noted that alternative embodiments of the devices, kits and methods of the present disclosure may not include all of the features described yet still benefit from at least some of the advantages of such features. Those of ordinary skill in the art may readily devise their own implementations of an apparatus and method that incorporate one or more of the features of the present disclosure and fall within the spirit and scope of the present disclosure.

1.-25. (canceled)

26. A self-stackable, tissue regenerating device comprising a receptacle, wherein the receptacle is polyhedral in shape, is meso-scale, micro-scale, or nano-scale, and is constructed from one or more solid, gelatinous, or viscous fluid biocompatible materials, wherein the one or more biocompatible materials form edges encompassing each polygonal face of the polyhedral shape.

27. The device of claim 26, wherein the polyhedral receptacle has a shape selected from the group consisting of a dodecahedron, a hexagonal prism, a hexagonal antiprism, a pentagonal dipyramid, and a tetrahedron.

28. The device of claim 26, wherein the device occupies a stable three dimensional volume and is not substantially deformed under biomechanical load within a range that is normal in a mammalian body and applied along multiple planes and axes.

29. The device of claim 26, wherein:
the interior of the receptacle is filled with the one or more biocompatible materials;
wherein the receptacle is partially enclosed, one or more of the polygonal faces of the polyhedral receptacle comprising the one or more biocompatible materials and the interior of the polyhedral receptacle being partially filled with the one or more biocompatible materials; or
all of the polygonal faces of the polyhedral receptacle and the interior of the polyhedral receptacle being empty and all the edges of the polygonal faces comprising the one or more biocompatible materials.

30. The device of claim 29, wherein the one or more biocompatible materials is selected from the group consisting of metal, alloy, ceramic, or plastic.

31. The device of claim 29, wherein the one or more biocompatible materials are sintered and porous.

32. The device of claim 29, wherein the one or more biocompatible materials are coated or adsorbed with one or of antimicrobial peptides, antibiotics, biomolecules, biologics, or nanostructures.

33. The device of claim 26, wherein the polyhedral receptacle comprises an empty interior or an interior partially filled with the one or more biocompatible materials and the empty or partially filled interior comprises: multiple finite compartments; a small rod internally, a small plate internally; a tenon protruding externally; or a mortise recessed into the one or more biocompatible materials.

34. The device of claim 26, wherein the polyhedral receptacle is capable of
being unfolded, or partially unfolded, into a flat polygonal net, a flat polygonal plate, or any other shape resulting from the folding or unfolding of the net or plate; or
being reversibly compressed.

35. The device of claim 26, further comprising, within the interior of the polyhedral receptacle, a biomimetic collagen construct.

36. The device of claim 35, wherein the device further comprises biofunctional cells.

37. The device of claim 36, wherein the biofunctional cells are one or more of fibroblasts, osteoclasts, osteocytes, chondrocytes, soft tissue cells, endothelial cells, blood cells, immune cells, or stem cells.

38. The device of claim 26, wherein the receptacle comprises a carrier for a slow-release drug, a delivery system for a slow-release drug, a medicament, one or more polymers, a glue, or one or more inorganic molecules.

39. The device of claim 26, wherein the device is one or more of: nano-constructed, capable of nano-assembly; capable of self-assembly; or self-replicating.

40. A composite device comprising a plurality of devices, each device being the device of claim 26.

41. The composite device of claim 40, wherein the plurality of devices are assembled or aggregated in a three-dimensional conformation in which each polyhedral shape of each device has some or all of its polygonal faces contiguous with or aligned with at least one polygonal face of a polyhedral shape of another device.

42. The composite device of claim 41, wherein there are no spaces between the devices.

43. The composite device of claim 41, wherein there are spaces between the devices.

44. The composite device of claim 41, wherein the devices are dense packed in three dimensions.

45. The composite device of claim 44, wherein the interior of the receptacles are empty and, as a result, the composite device comprises a complex network of compartments in three dimensions, the network comprising a container.

46. The composite device of claim 40, further comprising an exterior hull surrounding the plurality of devices.

47. The composite device of claim 46, wherein the exterior hull comprises a resorbable polymer.

48. The composite device of claim 46, wherein the devices are embedded, packed, or stacked within niches or recesses in the interior surface of the exterior hull.

49. The composite device of claim 40, wherein the devices are of more than one shape.

50. A method of manufacturing a device, the method comprising: providing a solid, gelatinous, or viscous fluid biocompatible material; and manufacturing the device of claim 26 from the biocompatible material.

51. The method of claim 50, wherein the manufacturing comprises selective laser melting (SLM).

52. The method of claim 50, wherein the manufacturing comprises rapid prototyping.

53. The method of claim 50, wherein the manufacturing comprises solid fabrication, selective laser sintering (SLS), extrusion, nano-assembly, nano-construction, or gel formation followed by hardening.

54. The method of claim 50, further comprising incorporating within the interior space of the polyhedral shape, a biomimetic collagen construct.

55. The method of claim 54, further comprising seeding biologically functional cells into the devices.

56. The method of claim 55, wherein the seeding occurs prior to manufacture of the device, after manufacture of the device but prior to implantation into a mammalian subject, or after manufacture and implantation of the device into a mammalian subject.

57. The method of claim 54, further comprising incorporating into the devices one or more of the TGF-β superfamily of ligands or one or more of BMP-1 family of proteases, the incorporating occurring prior to, or after, delivery of the device to a tissue in a mammalian subject.

58. A method of making a composite device, the method comprising:
providing a plurality of devices, each of which is the device of claim 26; and
assembling or aggregating the devices into a three dimensional conformation in which each polyhedral receptacle of each device has some or all of its polygonal faces contiguous with or aligned with at least one polygonal face of a polyhedral shape of another device.

59. The method of claim 58, wherein the assembly or aggregation occurs prior to delivery of the plurality of devices to a tissue in a mammalian subject.

60. The method of claim 58, wherein the assembly or aggregation occurs after delivery of the plurality of devices to a tissue in a mammalian subject.

61. A method of tissue regeneration, the method comprising:
providing a plurality of devices of claim 26; and
delivering the plurality of devices to a tissue in or on a mammalian subject, wherein the tissue is in need of regeneration.

62. The method of claim 61, wherein the plurality of devices are aggregated or assembled into a composite device prior to the delivery.

63. The method of claim 61, wherein the delivery comprises:
placing, projecting, pushing, driving, or embedding the plurality of devices directly into a tissue void;
infusing the plurality of devices in a discrete particulate flow through a catheter or channel;
introducing the plurality of devices into the body of the mammalian subject via the upper bowel, the lower bowel, the ureter, the urethra, or the vagina;
subcutaneous administration; or
intravenous administration.

64. The method of claim 61, wherein the mammalian subject is a human.

65. The method of claim 61, wherein the devices provide immediate structural stability to the tissue.

66. The method of claim 61, wherein the devices form a stably supported and immobilized three dimensional matrix in the tissue after the delivery.

67. The method of claim 61, wherein the tissue comprises bone.

68. The method of claim 61, wherein the tissue comprises cartilage.

69. The method of claim 61, wherein any of unfolding, refolding, compression, or decompression of the devices occurs before, during, or after a procedure to deliver the devices to the tissue.

70. The method of claim 61, wherein biofunctional cells are seeded into the devices prior to the delivery.

71. The method of claim 61, wherein the devices are seeded in vivo with the biofunctional cells after the delivery.

72. A kit comprising one or more of the devices of claim 26.

73. The kit of claim 72, further comprising one or more surgical instruments or other equipment for promoting tissue regeneration in vivo.
